SEQUENCE LISTING (9 sequences)

SEQ ID NO: 1 - protein, 10 aa, Homo sapiens
  26..35: N-terminal epitope target sequence in lysyl oxidase-like 2 enzyme
QYDSWPHYPE

SEQ ID NO: 2 - protein, 6 aa, Homo sapiens
  26..31: N-terminal epitope target sequence in lysyl oxidase-like 2 enzyme
QYDSWP

SEQ ID NO: 3 - protein, 11 aa, Artificial Sequence
  Elongated peptide
AQYDSWPHYPE

SEQ ID NO: 4 - protein, 10 aa, Artificial Sequence
  Biotinylated peptide with optional linker
  SITE 10: C-terminus is biotinylated with an optional linker between the C-terminus and the biotin
QYDSWPHYPE

SEQ ID NO: 5 - protein, 10 aa, Artificial Sequence
  calibrator peptide
QYDSWPHYPE

SEQ ID NO: 6 - protein, 10 aa, Artificial Sequence
  nonsense peptide
IKAPKLPGGY

SEQ ID NO: 7 - protein, 10 aa, Artificial Sequence
  nonsense coating peptide
  SITE 1: N-terminus is biotinylated
IKAPKLPGGY

SEQ ID NO: 8 - protein, 10 aa, Artificial Sequence
  Immunogenic peptide
  SITE 10: Keyhole Limpet Hemocyanin attached to C- terminus
QYDSWPHYPE

SEQ ID NO: 9 - protein, 10 aa, Artificial Sequence
  biotinylated coater peptide
  SITE 10: C-terminus biotinylated
QYDSWPHYPE

(19) United States
(12) Patent Application Publication
(10) Pub. No.: US 2020/0123272 A1
(43) Pub. Date: Apr. 23, 2020

(54) Lysyl Oxidase-Like 2 Assay

(71) Applicant: Nordic Bioscience A/S, Herlev (DK)

(72) Inventors: Diana Oersnes-Leeming, Klampenborg (DK); Morten Karsdal, København Ø (DK); Signe Holm Nielsen, Kobenhavn (DK)

(73) Assignee: Nordic Bioscience A/S, Herlev (DK)

(21) Appl. No.: 16/494,472

(22) PCT Filed: Mar. 14, 2018
(86) PCT No.: PCT/EP2018/056364

§ 371 Date: Sep. 16, 2019

(30) Foreign Application Priority Data
Mar. 16, 2017 (GB) 1704206.0

(51) Int. Cl. C07K 16/40; G01N 33/68

(52) U.S. Cl. CPC C07K 16/40; G01N 33/68; C07K 2317/33; C07K 2317/34; G01N 2333/902

(57) ABSTRACT

The present invention relates to an antibody, wherein the antibody specifically binds to the N-terminus of Lysyl Oxidase-Like 2 (LOXL2), and its use in a method of immunoassay for detecting or quantitating LOXL2 in a sample. The method may be used to evaluate LOXL2-associated diseases, and may also be used to evaluate the efficacy of anti-LOXL2 therapeutics.

### FIELD OF THE INVENTION

The present invention relates to an anti-LOXL2 antibody and its use in a method of immunoassay for detecting or quantitating LOXL2.

### BACKGROUND

Post translation modifications of the extracellular matrix (ECM) are important aspect of ECM maturation and cell signaling properties of proteins (1) and especially collagen and elastin crosslinking by the family of lysyl oxidase (LOX) has gained increasing attention in diseases leading to fibrosis in the pulmonary region and in cancer (2) (3) (4). LOXL2 is a member of the lysyl oxidase (LOX) gene family in which five LOX family genes have been identified in mammalian genomes encoding the LOX and LOX-like proteins 1 to 4 (LOXL1, LOXL2, LOXL3, and LOXL4 (5,6). The full members of the family have a conserved catalytic domain located at the COOH terminus, whereas the NH₂ terminus region of the LOX isoforms is more divergent and is thought to determine the individual role and tissue distribution of each isoenzyme (7). LOX family members are known to be responsible for normal and pathology relevant crosslinking generating increased tissue stiffness, which in uncontrolled state promotes resistance to fibrolysis in fibrotic tissue (8,9). LOX is overexpressed in the microenvironment of fibrotic lesions especially produced by activated fibroblasts (10), with focus on LOXL2 since this is the most highly expressed in advanced fibrosis stage out of nine members of the LOX family (8,11,12). LOX is a copper dependent pro-enzyme that is essential for the stabilization of the ECM, inducing cross-linking via the formation of lysine-derived cross-links, which may constitute an essential defense against the proteolysis executed by nonspecific proteases. LOX has the highest affinity for collagens precipitated in the form of fibrils and clustered forms of elastin (13).

LOXL2 activity and expression has been shown to be associated with cancer and fibrosis (9), including signalling roles in cancer proliferation and dedifferentiation, and increased stability of collagens accumulating during fibrosis (14-16). The number of cross-links per collagen molecule has been shown to be elevated in fibrotic tissue compared to healthy state (9). LOX is expressed in most tissues, however LOXL2 appears to be more linked to tissue affected by fibrosis and cancer and is associated with worsening of tumour grade and fibrosis stage (17). Therapeutic interventions targeting this enzyme and its cross-linking activity focusing on the inhibition fibrosis and cancer progression has been tested using a humanized monoclonal antibody (17), and a clinical study in patients with fibrotic non-alcoholic steatohepatitis and primary sclerosing cholangitis is ongoing (18,19). LOXL2 has been found to be over-expressed in lung tissue samples from idiopathic pulmonary fibrosis (IPF) patients, especially in tissues with activated fibroblasts, reactive pneumocytes and vasculature in fibroblast foci (8,12,17). This was demonstrated by a dramatic increase in LOX activity during the development of pulmonary fibrosis (8). Nevertheless, recently the lack of efficacy of a humanized monoclonal antibody employed in a phase III clinical trial including IPF patients was reported (20).

In order to evaluate and/or monitor the efficacy of new anti-LOXL2 therapeutics it is necessary to provide an assay that can accurately quantify the amount of LOXL2 in circulation.

Quantitative assessments of LOXL2 in circulation have been described previously. Chien et al. (Eur respire J (2014): 1430-1438) developed a sandwich assay based on a polyclonal antibody to full length recombinant human LOXL2 protein (R&D Systems) and a monoclonal antibody to the recombinant C-terminal catalytic domain. The same assay is disclosed in Chien, WO 2012/167181 (both utilize a rabbit anti-LOXL2 pAb and mAb AB0030). Whilst elevated levels of LOXL2 were reported in most subjects with IPF, approximately 90% of healthy subjects had no detectable LOXL2 in serum, i.e. had LOLX2 levels below the lower level of detection (LLOD). Similarly, the LOXL2 assay was unable to positively identify the presence of LOXL2 in serum samples of five of fifteen patients known to be suffering from IPF. The inability to quantitate low levels of circulating LOXL2 in healthy human subjects and in some subjects known to suffer from IPF indicates an inability to detect early pathological changes where the increase in concentration of LOXL2 above the normal background level is expected to be only very small. Furthermore, the concluding remarks in Chien et al note that although there is a relationship between higher LOXL2 levels and increased risk for poor IPF outcomes, the results obtained indicated that serum LOXL2 may not be a suitable biomarker for IPF.

There therefore remains a need for an assay that accurately quantifies LOXL2 in circulation and is sensitive enough to detect early pathological changes in LOXL2 serum levels where the increase in concentration of LOXL2 above the normal background level is expected to be only very small.

As such, the inventors of the instant application aimed to develop such an assay for quantifying LOXL2, with particular emphasis on quantifying LOXL2 levels at early stages of disease where biochemical changes in circulation are expected to be very small.

### SUMMARY OF THE INVENTION

In order to achieve this, the inventors developed an assay directed to the free N-terminal of LOXL2. An unexpectedly high level of circulating LOXL2 was detected in serum from human subjects using that assay, even in serum originating from healthy individuals, suggesting that it could allow detection of early pathological changes. Surprisingly, it was found that even a competition immunoassay directed to the free N-terminal of LOXL2, which is usually considered to be less sensitive than a sandwich immunoassay, was able to detect and quantify circulating levels of LOXL2 in healthy subjects; all samples from the healthy subjects had LOXL2 levels above the LLOD. The free N-terminal of LOXL2 would therefore appear to be a superior biomarker for evaluating circulating LOXL2 levels.

Accordingly, in a first aspect the present invention relates to an antibody, wherein the antibody specifically binds to the N-terminus of LOXL2.

Preferably, the antibody specifically binds to an N-terminal epitope comprised in the N-terminal amino acid sequence H₂N-QYDSWPHYPE (SEQ ID NO: 1). Preferably still, the antibody specifically binds to the N-terminal amino acid sequence H₂N-QYDSWP (SEQ ID NO: 2).

The antibody is preferably a monoclonal antibody.

Preferably, the antibody does not specifically recognise or bind an N-extended elongated version of said N-terminal amino acid sequence. In this regard “N-extended elongated version of said N-terminal amino acid sequence” means one or more amino acids extending beyond the N-terminus of the sequence H₂N-QYDSWPHYPE (SEQ ID NO: 1). For example, if the N-terminal amino acid sequence H₂N-QYDSWPHYPE (SEQ ID NO: 1) was elongated by an alanine residue then the corresponding “N-extended elongated version” would be H₂N-AQYDSWPHYPE . . . (SEQ ID NO: 3).

In a second aspect, the present invention relates to a method of immunoassay for detecting or quantitating in a sample LOXL2, wherein said method comprises contacting a sample comprising said LOXL2 with an antibody as described supra, and determining the amount of binding of said antibody.

Preferably, the sample is a biofluid, such as, but not limited to, serum, plasma, urine or amniotic fluid.

The immunoassay may be a competition assay or a sandwich assay. The immunoassay may be, but is not limited to, a radioimmunoassay or an enzyme-linked immunosorbent assay.

The method of immunoassay may further comprise the step of correlating the quantity of LOXL2 determined by said method with standard LOXL2-associated disease samples of known disease severity to evaluate the severity of said LOXL2-associated disease. Such LOXL2-associated diseases may be, but are not limited to, fibrosis, such as IPF or Chronic Obstructive Pulmonary Disease (COPD), or cancer, such as non-small cell lung cancer (NSCLC), small cell lung cancer (SCLC), colon cancer, melanoma, ovarian cancer, pancreatic cancer, prostate cancer, or breast cancer.

The method of immunoassay may also be used in the evaluation of the efficacy of novel anti-LOXL2 therapeutics. In this regard, the method further comprises quantifying the amount of LOXL2 in at least two biological samples obtained from a subject at a first time point and at at least one subsequent time point during a period of administration of the anti-LOXL2 therapeutic to the subject. A reduction in the quantity of LOXL2 from the first time point to the at least one subsequent time point during the period of administration of the anti-LOXL2 therapeutic is indicative of an efficacious anti-LOXL2 therapeutic.

In this regard the “anti-LOXL2 therapeutic” may be, but is not limited to, small molecule LOXL2-antagonist drugs or biosimilars (e.g. monoclonal antibody therapy) that target and reduce the quantity of LOXL2 in a subject.

The present invention is directed further to a kit for use in the immunoassay as described herein. The kit comprises an antibody as described supra and at least one of:
a streptavidin coated 96 well plate
a peptide which is reactive with said antibody, which may be a biotinylated peptide H₂N-QYDSWPHYPE-L-Biotin (SEQ ID NO: 4), wherein L is an optional linker
an optionally biotinylated secondary antibody for use in a sandwich immunoassay
a calibrator peptide comprising the N-terminal sequence H₂N-QYDSWPHYPE . . . (SEQ ID NO: 5)
an antibody HRP labelling kit
an antibody radiolabeling kit
an assay visualization kit.

### DESCRIPTION OF THE FIGURES

FIG. 1. Specificity of the LOXL2 ELISA: The activity of the monoclonal antibody employed in the LOXL2 ELISA towards the target peptide (QYDSWPHYPE; SEQ ID NO: 1), the elongated peptide (AQYDSWPHYPE; SEQ ID NO: 3), a non-sense peptide (IKAPKLPGGY; SEQ ID NO: 6) and a non-sense coating peptide (biotin-IKAPKLPGGY; SEQ ID NO: 7). Reactivity was shown as percent inhibition of the zero sample (buffer) signal assessed as optical density (OD) at 450 nm (subtracted the background at 650 nm) and as a function of the peptide concentrations.

FIG. 2. Reactivity towards recombinant LOXL2: Recombinant LOXL2 (rLOXL2) was added in the indicated concentrations to the LOXL2 ELISA. The LOXL2 ELISA data are shown as mean of a double determinations for each rLOXL2 concentration.

FIG. 3. LOXL2 was assessed in serum of patients included in two different cohorts. Cohort 1 (FIG. 3A) included patients with breast cancer (n=20), colon cancer (n=7), gastric cancer (n=8), melanoma (n=19), NSCLC (n=8), SCLS (n=7), ovarian cancer (n=9), pancreatic cancer (n=5) or prostate cancer (n=14). Data were compared using Kruskal-Willis comparison test for non-parametric data. Data are shown as box-whisker plots. Significance levels: ***: p<0.001 and ****: p<0.0001. Cohort 2 (FIG. 3B) included serum samples from patients diagnosed with IPF (n=120) and healthy controls (n=51). Groups were compared using unpaired, two-tailed Mann-Whitney test. Data are shown as box-whisker plots. Significance levels: ***: p<0.001 and ****: p<0.0001.

### EXAMPLES

The presently disclosed embodiments is described in the following Examples, which are set forth to aid in the understanding of the disclosure, and should not be construed to limit in any way the scope of the disclosure as defined in the claims which follow thereafter. The following examples are put forth so as to provide those of ordinary skill in the art with a complete disclosure and description of how to make and use the described embodiments, and are not intended to limit the scope of the present disclosure nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

In the following examples, the following materials and methods were employed.

### Selection of Peptides

In the “Uniprot.org” database the N-terminal cleavage site separating the signal peptide from LOXL2 (Human LOXL2, UniProtKB Q9Y4K0) is predicted at amino acid position 25′. To generate an antibody specific for this clevage site (↓) a ten amino acid sequence adjacent to the signal peptide cleavage site was chosen as the target: 26′ ↓QYDSWPHYPE′ 35. This sequence was used for immunization of mice and used as the standard peptide in assay development. The sequence was BLASTed for species homology and homology to other human secreted extracellular matrix proteins using the Prabi-Lyon-Gerland “NPS@: Network Protein Sequence Analysis with the UniprotKB/Swiss-prot database” software online (4).

Synthetic peptides used for monoclonal antibody production and validation of the ELISA assay were purchased from Chinese Peptide Company (China) and Genscript (Picataway, N.J., USA). A biotinylated peptide (QYDSWPHYPE-biotin) was included as a coating peptide on streptavidin-coated ELISA plates. The specificity of the antibody was tested by including an elongated selection peptide with an additional amino acid added to the N-terminal of the target peptide sequence (AQYDSWPHYPE; SEQ ID NO: 1), as well as a non-sense selection peptide (IKAPKLPGGY; SEQ ID NO: 6) and a non-sense biotinylated coating peptide (biotin-IKAPKLPGGY; SEQ ID NO: 7) in the assay validation. The immunogenic peptide (QYDSWPHYPE-KLH; SEQ ID NO: 8) was generated by covalently cross-linking the selection peptide to Keyhole Limpet Hempcyanin (KLH) carrier protein using Succinimidyl 4-(N-maleimidomethyl)cyclohexane-1-carboxylate, SMCC (Thermo Scientific, Waltham, Mass., USA, cat.no. 22336).

### Monoclonal Antibody Production

Four to six weeks old Balb/C mice were immunized by subcutaneous injection of 200 μL emulsified antigen and 50 μg immunogenic peptide (QYDSWPHYPE-KLH; SEQ ID NO: 8) mixed with Freund's incomplete adjuvant (Sigma-Aldrich, St. Louis, Mo., USA). Consecutive immunizations were performed at 2-weeks interval until stable sera titer levels were reached. The mouse with the highest titer rested for four weeks were boosted with 50 pg immunogenic peptide in 100 μL 0.9% NaCl solution intravenously. Hybridoma cells were produced by fusing spleen cells with SP2/0 myeloma cells as previously described (Gefter M L, Margulies D H, Scharff M D. A simple method for polyethylene glycol-promoted hybridization of mouse myeloma cells. Somatic Cell Genet. 1977;3:231-6). The resultant hybridoma cells were cultured in 96-well microtiter plates and standard limited dilution was used to secure monoclonal growth. The supernatants were screened for reactivity using the biotinylated coater peptide (QYDSWPHYPE-biotin; SEQ ID NO: 9).

### Clone Characterization

Native reactivity and peptide affinity of the monoclonal antibodies were evaluated by displacement using human serum samples and the selection /standard peptide (QYDSWPHYPE; SEQ ID NO: 1) in a preliminary ELISA using 10 ng/mL biotinylated coating peptide on streptavidin-coated microtiter plates (Roche, Basel, Switzerland, cat. #11940279) and the supernatant from the antibody producing monoclonal hybridoma cells. The clones with best peptide and native reactivity were purified using protein-G-columns according to the manufacturer's instructions (GE Healthcare Life Sciences, Little Chalfont, UK, cat. #17-0404-01). The final selection of the monoclonal antibody for assay development and validation was based on high reactivity towards the selection/standard peptide, relevant native samples including human serum samples and recombinant LOXL2, homolog2 (RnD system, Cat no. 2639-AO).

### LOXL2 ELISA Protocol

Optimal incubation-buffer, -time and -temperature, as well as the optimal concentrations of antibody and coating peptide were determined and the finalized LOXL2 competitive ELISA protocol was developed to the following protocol:

A 96-well streptavidin-coated microtiter plate was coated with 0.5 ng/mL biotinylated coating peptide diluted in assay buffer (25 mM TBS-BTB, 4 g/L NaCl, pH 7.4) and incubated for 30 min. at 20° C. shaking (300 rpm) in darkness. 20 μL standard peptide (500 ng/mL) or pre-diluted serum sample (1:2) were added to appropriate wells, followed by the addition of 100 μL monoclonal antibody dissolved in assay buffer to a concentration of 14 ng/mL to each well and incubated 1 hour at 20° C. shaking (300 rpm) in darkness. 100 μL of rabbit POD-conjugated anti-mouse IgG antibody (Jackson Immunoresearch Laboratories, Pa., USA, Cat no. 119936) diluted 1:5000 in assay buffer was added to each well and incubated 1 hour at 20° C. shaking (300 rpm) in darkness. All incubation steps were followed by five washes in washing buffer (20 mM Tris, 50 mM NaCl, pH 7.2). Finally, 100 μL tetramethylbenzinidine (TMB) (cat. 4380H, Kem-En-Tec Diagnostics, Denmark) was added to each well and the plate was incubated for 15 minutes at 20° C. in darkness shaking (300 rpm). The enzymatic reaction was stopped by adding 0.18 M H2SO4 and absorbance was measured at 450 nm with 650 nm as reference. A calibration curve was plotted using a 4-parameter logistic curve fit. Data were analyzed using the SoftMax Pro v.6.3 software.

### Technical Evaluation of the LOXL2 ELISA

The inter- and intra-assay variation was determined by ten independent runs including eight quality control samples and two internal controls covering the detection range, with each run consisting of double-determinations of the samples. Five quality control samples consisted of: four human serum samples of which two where spiked with the synthetic specific peptide, and one sample of buffer spiked with the synthetic specific peptide. Intra-assay variation was calculated as the mean coefficient of variance (CV %) within plates and the inter-assay variation was calculated as the mean CV % between the ten individual runs. Two-fold dilutions of three human serum samples were used to calculate linearity. Recovery percentages were calculated with the un-diluted, 1:2 or 1:4 sample as a reference value. The lower limit of detection (LLOD) was determined from 21 measurements of the zero sample (assay buffer) and was calculated as the mean+three standard deviations. The upper limit of detection (ULOD) was determined from ten independent runs of the highest standard peptide concentration and was calculated as the mean back-calibration calculation+three standard deviations. The lower limit of quantification (LLOQ) was determined from three independent runs of a serum sample diluted stepwise and calculated as the highest LOXL2 level quantifiable in serum with a coefficient of variation below 30%. Analyte stability was first determined by the effect of repeated freeze/thaw of serum samples by measuring the LOXL2 level in three human serum samples in four freeze/thaw cycles. The freeze/thaw recovery was calculated with the zero cycle as reference. Second, analyte stability was determined at different time points and temperatures by measuring LOXL2 level in three human serum samples after 0, 2, 4, 24 and 48 hours of storage at either 4° C. or 20° C. Recovery was calculated with 0 hours as reference. Interference was determined by adding a low/high content of hemoglobin (0.155/0.310 mM), lipemia/lipids (4.83/10.98 mM) and biotin (30/90 ng/mL) to a serum sample of known concentration. Recovery percentage was calculated with the normal serum sample as reference.

### Clinical Validation of LOXL2—Patient Serum Samples

Patient serum samples in cohort 1 included malignant melanoma, breast-, colon-, lung-, ovarian-, pancreatic-, prostate cancer and colonoscopy-negative controls. The cohort was obtained from the commercial vendors Proteogenex (Culver City, Calif., USA) and Asterand Bioscience (Hertfordshire, UK). Cohort 2 included serum samples from patients diagnosed with IPF (baseline samples, CTgov reg. NCT00786201) and healthy control serum samples acquired from the commercial vendor Valley Biomedical (Winchester, Va., USA). Details of the studies are shown in table 1. All studies received suitable Institutional Review Board/Independent Ethical Committee approved sample collection including patients filed informed consent.

TABLE 1
Overview of clinical and patients demographics of
cohort 1 and 2.

| | | No. of | Mean age | Gender |
|---|---|---|---|---|
| Cohort | Samples | subjects | years (SD) | % females |
| 1 | Colonoscopy- | 16 | 55.5 (5.6) | 44 |
| | negative controls | | | |
| 1 | NSCLC patients | 19 | 60.4 (9.3) | 21 |
| 1 | SCLC patients | 7 | 61.4 (12.6) | 29 |
| 1 | Breast cancer | 20 | 55 (10.3) | 95 |
| | patients | | | |
| 1 | Colon cancer | 7 | 61.9 (8.6) | 86 |
| | patients | | | |
| 1 | Gastric cancer | 8 | 69.3 (9.2) | 38 |
| | patients | | | |
| 1 | Melanoma patients | 7 | 45.6 (14.4) | 43 |
| 1 | Ovary cancer | 9 | 55.9 (11.2) | 100 |
| 1 | Pancreas cancer | 5 | 68.4 (10.1) | 60 |
| 1 | Prostate cancer | 14 | 63.7 (5.7) | 0 |
| 2 | IPF patients | 124 | 65 (8.1) | 20 |
| 2 | Healthy controls | 51 | 35.9 (11.9) | 19 |

SD: Standard deviation.
SCLC: small cell lung cancer;
NSCLC: non-SCLC.

### Statistical Analysis

The level of LOXL2 in serum samples was compared using one-way ANOVA adjusted for Tukey's multiple comparisons test (parametric data), Kruskal-Wallis adjusted for Dunn's multiple comparisons test (non-parametric data) or unpaired, two-tailed Mann-Whitney test. The diagnostic power was investigated by the area under the receiver operating characteristics (AUROC). Sensitivity and specificity were determined for optimal cut-off values based on the ROC curves. P-values <0.05 were considered significant. Graphs and statistical analyses were performed using GraphPad Prism version 6 (GraphPad Software, Inc., Calif., USA) or and MedCalc Statistical Software version 12 (MedCalc Software, Ostend, Belgium).

### RESULTS

### Selection and Specificity of the Antibody Utilized in the LOXL2 Assay

The hybridoma clone producing monoclonal antibodies with the best native reactivity towards human serum samples and affinity towards the selection peptide was selected for ELISA optimization and validation.

The N-terminal site of the human target sequence, ↓26′QYDSWPHYPE′35, was selected as the signal peptide cleavage (↓). The sequence blast showing a 100% homology to Pongo Abelii and 80% homology to Bos Taurus with a mismatch at amino acid position three and six from the signal peptide cleavage site. The protein blast did not indicate that other similar sequences could be found in other proteins and serve as potential cross-reacting proteins in the assay.

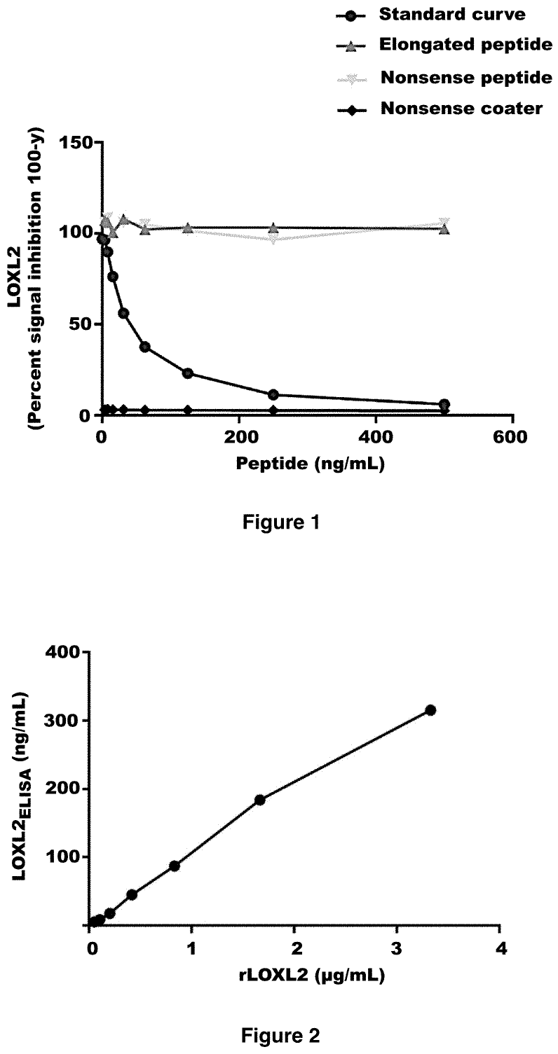

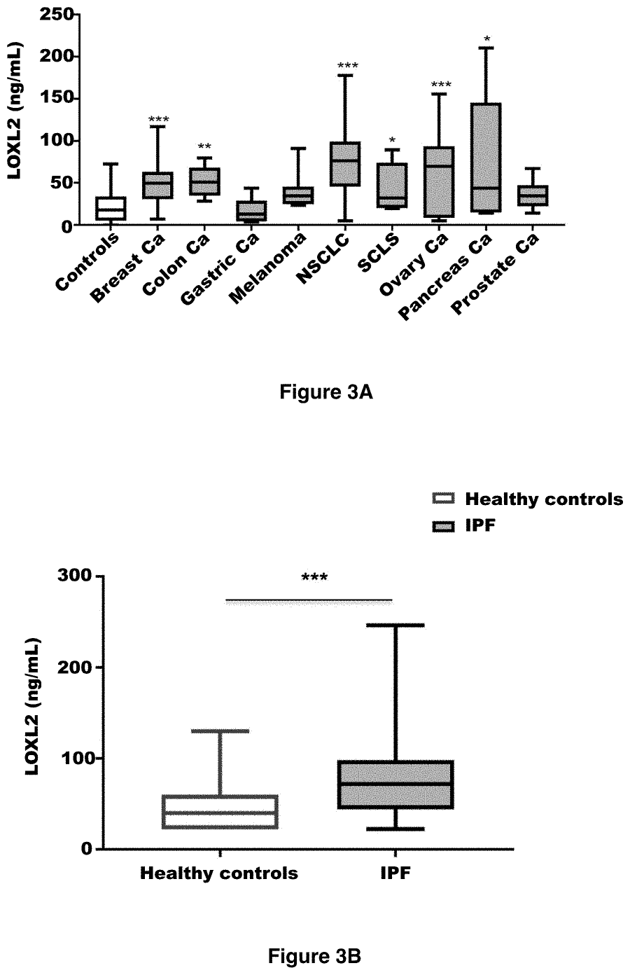

The specificity of the competitive LOXL2 ELISA assay was evaluated by analyzing the reactivity towards the target (selection) peptide, a non-sense peptide, an elongated peptide and using a non-sense biotinylated coating peptide; data are shown in FIG. 1. The antibody reacted towards the selection peptide generating a standard curve following a five parametric curve. No detectable signal was observed using a non-sense biotinylated coating peptide, elongated peptide or non-sense peptide. These data suggest that the selected antibody was specific towards the target epitope. Furthermore, test of the recombinant LOXL2 protein without the signal peptide showed that the antibody was able to react towards this protein (FIG. 2).

### Technical Evaluation of the LOXL2 ELISA Assay

A range of technical validations were performed to evaluate the LOXL2 ELISA assay. The measuring range (LLOD to ULOD) of the assay was determined to 5.8-401.5 ng/mL and the lower limit of quantification (LLOQ) was 12.9 ng/mL. The intra- and inter-assay variation was 8% and 12%, respectively, and within our acceptance criteria <10% for the intra-assay variation and <15% for the inter-assay variation. The recommended human serum dilution was 1:2 and linearity was within +/−120%. The mean analyte recovery in three serum was 106% after 4 freeze/thaw cycles and after storage at 4° C. for 2-48 hours the recovery was between 83-99% or at 20° C. for 2-48 hours the recovery was between 84-97%. The acceptance criterion was a recovery within 100%±20%. These data indicate that the analyte in serum is highly stable at 4 and 20° C. No interference was detected from either low or high contents of lipids or hemoglobin with recoveries ranging from 85-112%. The acceptance criterion was a recovery within 100%±20%. For biotin the high level of biotin reduced the levels of LOXL2 whereas low levels (30 ng/mL) did not affect the assay. Thus the cutoff level should be biotin below 30 ng/mL.

TABLE 2
Summary of the technical details of the LOXL2 ELISA

| Technical validation step | LOXL2 performance |
|---|---|
| Detection range (LLOD-ULOD) | 5.7-401.5 ng/mL |
| Lower limit of quantification (LLOQ) | 2.9 ng/mL |
| Intra-assay variation | 8% |
| Inter-assay variation | 12% |
| Dilution of serum samples | 1:2 |
| Dilution recovery¹ | 102% (95-109%) |
| Freeze/thaw recovery (4 cycles)¹ | 106% (104-108%) |
| Analyte stability up to 48 h, 4° C.¹ | 84% (84-97%) |
| Analyte stability up to 48 h, 20° C.¹ | 93% (83-99%) |
| Interference Lipids, low/high | 106%/85% |
| Interference Biotin, low/high | 95%/21% |
| Interference Hemoglobin, low/high | 98%/112% |

¹Percentages are reported as mean with range shown in brackets

LOXL2 is Elevated in Patients with Cancer and IPF

Serum LOXL2 in cohort 1 was elevated 218, 227, 375, 213, 225 and 325% in patients with Breast Cancer, colon cancer, NSCLC, SCLS, ovarian cancer and pancreatic cancer, respectively, compared to healthy controls (p=0.05-0.0001). Furthermore, it was found that serum LOXL2 was highly elevated in patients with IPF, showing a 163% increase in IPF patients compared to healthy controls (p<0.0001). The diagnostic value of LOXL2 for assessed by the AUC was 0.89 for diagnosis of NSCLS (p<0.0001), and 0.72 (p=0.02) and 0.81 (p<0.0001) for diagnosis of prostate cancer (PCa) and breast cancer (BCa), respectively. The diagnostic value of LOXL2 for diagnosis of IPF versus healthy controls was AUC of 0.72 (p<0.0001) (Table 3).

TABLE 3
AUROC for diagnosis of IPF or cancer by LOXL2,
patient group with n > 10.

| | | | | | Cut |
|---|---|---|---|---|---|
| | | | | | off |
| Disease | AUC | Sensitivity | Specificity | P-value | ng/mL |
| NSCLC | 0.89 | 73.7 | 93.7 | <0.0001 | 51.4 |
| BCa | 0.81 | 70.0 | 81.2 | <0.0001 | 34.3 |
| PCa | 0.72 | 85.7 | 62.5 | 0.02 | 19.8 |
| IPF | 0.72 | 52.5 | 88.2 | <0.0001 | 69.0 |

### Discussion

The present invention provides a novel assay for the detection of an N-terminal neoepitope of human LOXL2. The assay was technically robust, with low limit of detection, acceptable intra- and inter-variation and linearity, interference and analyte stability at 20- and 4° C. It was also shown that using this newly developed LOXL2 assay, LOXL2 levels were higher in fibrosis related disease such as IPF and lung cancer. Furthermore, the diagnostic value of LOX2 for NCSLC and IPF was high, with AUROC ranging from 0.72-0.89. Finally, this newly developed assay was able to detect LOXL2 in healthy individuals.

In conclusion, the present invention provides a technically robust assay for a neo-epitope site in LOXL2 at the N-terminal site and showed that levels of LOXL2 were elevated in patients with cancer and fibrosis related disease. Thus, we demonstrated the feasibility and the potential biological value of this newly developed LOXL2 assay.

It is envisaged that this assay may be a useful tool in the evaluation of novel anti-LOXL2 therapeutics.

In this specification, unless expressly otherwise indicated, the word ‘or’ is used in the sense of an operator that returns a true value when either or both of the stated conditions is met, as opposed to the operator ‘exclusive or’ which requires that only one of the conditions is met. The word ‘comprising’ is used in the sense of ‘including’ rather than in to mean ‘consisting of’. All prior teachings acknowledged above are hereby incorporated by reference. No acknowledgement of any prior published document herein should be taken to be an admission or representation that the teaching thereof was common general knowledge in Australia or elsewhere at the date hereof.

### REFERENCES

1. Karsdal M A, Henriksen K, Leeming D J, Woodworth T, Vassiliadis E, Bay-Jensen A C. Novel combinations of Post-Translational Modification (PTM) neo-epitopes provide tissue-specific biochemical markers-are they the cause or the consequence of the disease? Clin. Biochem. 2010. page 793-804.

2. Leeming D J, Bay-Jensen A C, Vassiliadis E, Larsen M R, Henriksen K, Karsdal M A. Post-translational modifications of the extracellular matrix are key events in cancer progression: opportunities for biochemical marker development. Biomarkers. Nordic Bioscience A/S, Herlev Hovedgade 207, Herlev, Denmark; 2011;16:193-205.

3. Kristensen J H, Karsdal M A, Genovese F, Johnson S, Svensson B, Jacobsen S, et al. The role of extracellular matrix quality in pulmonary fibrosis. Respiration [Internet]. 2014;88:487-99. Available from: www.ncbi.nlm.nih.gov/pubmed/25359084

4. Peinado H, Moreno-Bueno G, Hardisson D, Perez-Gomez E, Santos V, Mendiola M, et al. Lysyl Oxidase-Like 2 as a New Poor Prognosis Marker of Squamous Cell Carcinomas. Cancer Res [Internet]. 2008 [cited 2017 Feb. 24];68:4541-50. Available from: www.ncbi.nlm.nih.gov/pubmed/18559498

5. Maki J M, Kivirikko K I. Cloning and characterization of a fourth human lysyl oxidase isoenzyme. Biochem J [Internet]. 2001 [cited 2017 Feb. 24];355:381-7. Available from: www.ncbi.nlm.nih.gov/pubmed/11284725

6. Csiszar K. Lysyl oxidases: a novel multifunctional amine oxidase family. Prog Nucleic Acid Res Mol Biol [Internet]. 2001 [cited 2017 Feb. 24];70:1-32. Available from: www.ncbi.nlm.nih.gov/pubmed/11642359

7. Molnar J, Fong K S K, He Q P, Hayashi K, Kim Y, Fong S F T, et al. Structural and functional diversity of lysyl oxidase and the LOX-like proteins. Biochim Biophys Acta [Internet]. 2003 [cited 2017 Feb. 24];1647:220-4. Available from: www.ncbi.nlm.nih.gov/pubmed/12686136

8. Counts D F, Evans J N, Dipetrillo T A, Sterling K M, Kelley J. Collagen lysyl oxidase activity in the lung increases during bleomycin-induced lung fibrosis. J Pharmacol Exp Ther [Internet]. 1981 [cited 2017 Feb. 28];219:675-8. Available from: www.ncbi.nlm.nih.gov/pubmed/6170751

9. Brinckmann J, Gaber Y, Gebker R, Neess C M, Sobhi H, Notbohm H, et al. Different Pattern of Collagen Cross-Links in Two Sclerotic Skin Diseases: Lipodermatosclerosis and Circumscribed Scleroderma. J Invest Dermatol [Internet]. 2001 [cited 2017 Feb. 28];117:269-73. Available from: www.ncbi.nlm.nih.gov/pubmed/11511304

10. Wong C C-L, Tse A P-W, Huang Y-P, Zhu Y-T, Chiu D K-C, Lai R K-H, et al. Lysyl oxidase-like 2 is critical to tumor microenvironment and metastatic niche formation in hepatocellular carcinoma. Hepatology [Internet]. 2014 [cited 2017 Feb. 28];60:1645-58. Available from: doi.wiley.com/10.1002/hep.27320

11. Iismaa S E, Mearns B M, Lorand L, Graham R M. Transglutaminases and disease: lessons from genetically engineered mouse models and inherited disorders. Physiol Rev [Internet]. 2009 [cited 2017 Feb. 28];89:991-1023. Available from: physrev.physiology.org/cgi/doi/10.1152/physrev.00044. 2008

12. Tzortzaki E G, Tischfield J A, Sahota A, Siafakas N M, Gordon M K, Gerecke D R. Expression of FACIT collagens XII and XIV during bleomycin-induced pulmonary fibrosis in mice. Anat Rec [Internet]. 2003 [cited 2017 Feb. 28];275A:1073-80. Available from: www.ncbi.nlm.nih.gov/pubmed/14613307

13. Cronlund A L, Smith B D, Kagan H M. Binding of lysyl oxidase to fibrils of type I collagen. Connect Tissue Res [Internet]. 1985 [cited 2017 Feb. 28];14:109-19. Available from: www.ncbi.nlm.nih.gov/pubmed/2866930

14. Wang Q, Wang Y, Hyde D M, Gotwals P J, Koteliansky V E, Ryan S T, et al. Reduction of bleomycin induced lung fibrosis by transforming growth factor beta soluble receptor in hamsters. Thorax [Internet]. 1999 [cited 2017 Feb. 28];54:805-12. Available from: www.ncbi.nlm.nih.gov/pubmed/10456973

15. Mutsaers S E, Marshall R P, Goldsack N R, Laurent G J, McAnulty R J. Effect of endothelin receptor antagonists (BQ-485, Ro 47-0203) on collagen deposition during the development of bleomycin-induced pulmonary fibrosis in rats. Pulm Pharmacol Ther [Internet]. 1998 [cited 2017 Feb. 28];11:221-5. Available from: linkinghub.elsevier.com/retrieve/pii/S1094553998901420

16. Borkham-Kamphorst E, Stoll D, Gressner A M, Weiskirchen R. Antisense strategy against PDGF B-chain proves effective in preventing experimental liver fibrogenesis. Biochem Biophys Res Commun [Internet]. 2004 [cited 2017 Feb. 28];321:413-23. Available from: www.ncbi.nlm.nih.gov/pubmed/15358192

17. Barry-Hamilton V, Spangler R, Marshall D, McCauley S, Rodriguez H M, Oyasu M, et al. Allosteric inhibition of lysyl oxidase-like-2 impedes the development of a pathologic microenvironment. Nat Med [Internet]. 2010 [cited 2017 Feb. 28];16:1009-17. Available from: www.nature.com/doifinder/10.1038/nm.2208

18. Romero-Chapman N, Lee J, Tinker D, Uriu-Hare J Y, Keen C L, Rucker R R. Purification, properties and influence of dietary copper on accumulation and functional activity of lysyl oxidase in rat skin. Biochem J [Internet]. 1991 [cited 2017 Feb. 28];657-62. Available from: www.ncbi.nlm.nih.gov/pubmed/1674861

19. Rucker R B, Romero-Chapman N, Wong T, Lee J, Steinberg F M, McGee C, et al. Modulation of lysyl oxidase by dietary copper in rats. J Nutr [Internet]. 1996 [cited 2017 Feb. 28];126:51-60. Available from: www.ncbi.nlm.nih.gov/pubmed/8558325

20. Raghu G, Brown K K, Collard H R, Cottin V, Gibson K F, Kaner R J, et al. Efficacy of simtuzumab versus placebo in patients with idiopathic pulmonary fibrosis: a randomised, double-blind, controlled, phase 2 trial. Lancet Respir Med [Internet]. 2017 [cited 2017 Feb. 28];5:22-32. Available from: www.ncbi.nlm.nih.gov/pubmed/27939076

1. An antibody, wherein the antibody specifically binds to the N-terminus of Lysyl Oxidase Like-2 (LOXL2).

2. The antibody according to claim 1, wherein the antibody specifically binds to the N-terminus amino acid sequence H₂N-QYDSWPHYPE (SEQ ID NO: 1).

3. The antibody according to claim 2, wherein the antibody does not specifically recognise or bind an N-extended elongated version of said N-terminal amino acid sequence.

4. The antibody according to claim 1, wherein the antibody is a monoclonal antibody.

5. A method of immunoassay for detecting or quantitating in a sample LOXL2, wherein said method comprises contacting a sample comprising said LOXL2 with an antibody that specifically binds to the N-terminus of LOXL2, and determining the amount of binding of said antibody.

6. The method according to claim 5, wherein the sample is a biofluid.

7. The method according to claim 6, wherein the biofluid is serum, plasma, urine or amniotic fluid.

8. The method according to claim 5, wherein the immunoassay is a competition immunoassay or a sandwich immunoassay.

9. The method according to claim 5, wherein the immunoassay is a radioimmunoassay or an enzyme-linked immunosorbent assay.

10. The method according to claim 5, wherein the method further comprises the step of correlating the quantity of LOXL2 determined by said method with standard LOXL2-associated disease samples of known disease severity to evaluate the severity of said LOXL2-associated disease.

11. The method according to claim 10, wherein the LOXL2-associated disease is fibrosis or cancer.

12. The method according to claim 10, wherein the LOXL2 associated disease is idiopathic pulmonary fibrosis (IPF), Chronic Obstructive Pulmonary Disease (COPD), non-small cell lung cancer (NSCLC), small cell lung cancer (SCLC), colon cancer, melanoma, ovarian cancer, pancreatic cancer, prostate cancer, or breast cancer.

13. A method for evaluating the efficacy of anti-LOXL2 therapeutics, wherein said method comprises using a method of immunoassay for quantitating in a sample LOXL2 to quantify the amount of LOXL2 in at least two samples, said samples having been obtained from a subject at a first time point and at at least one subsequent time point during a period of administration of the anti-LOXL2 therapeutic to said subject, wherein the method of immunoassay comprises contacting each sample with an antibody that specifically binds to the N-terminus of LOXL2 and determining the amount of binding of said antibody, and wherein a reduction in the quantity of LOXL2 from said first time point to said at least one subsequent time point during the period of administration of the anti-LOXL2 therapeutic is indicative of an efficacious anti-LOXL2 therapeutic.

14. The method according to claim 13, wherein the sample is a biofluid.

15. The method according to claim 14, wherein the biofluid is serum, plasma, urine or amniotic fluid.

16. The method according to claim 13, wherein the anti-LOXL2 therapeutic is a small molecule LOXL2-antagonist drug or a biosimilar that targets and reduces the quantity of LOXL2 in a subject.

17. A kit for use in a method of immunoassay for detecting or quantitating in a sample LOXL2, wherein the kit comprises an antibody that specifically binds to the N-terminus of LOL2, as and at least one of:
a streptavidin coated 96 well plate
a peptide which is reactive with said antibody, which may be a biotinylated peptide H₂N-QYDSWPHYPE-L-Biotin (SEQ ID NO: 4), wherein L is an optional linker
an optionally biotinylated secondary antibody for use in a sandwich immunoassay
a calibrator peptide comprising the N-terminal sequence H₂N-QYDSWPHYPE . . . (SEQ ID NO: 5)
an antibody HRP labelling kit
an antibody radiolabeling kit
an assay visualization kit.
